(19) United States
(12) Patent Application Publication
(10) Pub. No.: US 2014/0193469 A1
(43) Pub. Date: Jul. 10, 2014

(54) AQUEOUS COMPOSITIONS COMPRISING POLYMER PARTICLES AND LOW LEVELS OF CLAY

(71) Applicant: JOHNSON & JOHNSON CONSUMER COMPANIES, INC., Skillman, NJ (US)

(72) Inventors: Theresa Chen, Princeton, NJ (US); Christina Irene Lee, Plainsboro, NJ (US); Jipsha Thakrar, Westampton, NJ (US); Anne-Sophie Brillouet, Pennington, NJ (US); Marisa DeVita Dufort, Hillsborough, NJ (US)

(21) Appl. No.: 13/738,001

(22) Filed: Jan. 10, 2013

(51) Int. Cl. A61K 8/26; A61K 8/81; A61Q 19/08; A61K 8/85

(52) U.S. Cl. CPC A61K 8/26; A61K 8/85; A61K 8/8152; A61Q 19/08
USPC 424/401; 424/78.03

(57) ABSTRACT

The present invention features a composition that includes about 0.75% to about 1.25% by weight of a clay portion that comprises bentonite. The composition includes polymer particles having an average particle size of less than about 20 microns and a refractive index of about 1.3 to about 1.4. The composition is substantially free of hydrophobic compounds. The composition is useful for treating under eye skin.

### FIELD OF THE INVENTION

The present invention relates to a composition comprising polymer particles, a clay and methods of use thereof

### BACKGROUND OF THE INVENTION

Wrinkles, puffiness, and sagging, particularly related to skin under the eyes are problematic signs of aging. While it is known to apply various compositions to skin under the eyes, various drawbacks still remain. In particular, the inventors have recognized that it would be desirable to reduce the appearance of the above signs of under eye skin aging in a manner that provides a pleasant tightening sensation to skin under the eyes. Even more importantly, the inventors have recognized that it is important to provide these benefits without introducing negative perceptions, such as over-tightening or undesirable flaking of the composition.

Accordingly, the inventors have now discovered that a particular composition that includes about 0.75% to about 1.25% by weight of a clay portion comprising bentonite and polymer particles. The polymer particles have an average particle size of less than about 20 microns and a refractive index of about 1.3 to about 1.4. The composition provides an improved appearance in a comfortable manner with reduced flaking.

### SUMMARY OF THE INVENTION

The present invention features a composition that comprises about 0.75% to about 1.25% by weight of a clay portion, wherein the clay portion comprises bentonite. The composition further comprises polymer particles that have an average particle size of less than about 20 microns and a refractive index of about 1.3 to about 1.4. The composition is substantially free of hydrophobic compounds. The composition is particularly useful for treating skin under the eyes.

### DETAILED DESCRIPTION OF THE INVENTION

Unless defined otherwise, all technical and scientific terms used herein have the meaning commonly understood by one of ordinary skill in the art to which the invention pertains. All publications, patent applications, patents, and other references mentioned herein are incorporated by reference in their entirety. Unless otherwise indicated, a percentage refers to a percentage by weight (i.e., %(W/W)).

As used herein, “signs of skin aging” means the presence of lines and wrinkles. As used herein, “wrinkle” means fine lines, fine wrinkles, or coarse wrinkles. Examples of wrinkles include, but are not limited to, fine lines around the eyes (e.g., “crow's feet”), forehead and cheek wrinkles, frown-lines, and laugh-lines around the mouth.

As used herein, “substantially free” means less than about 2% by weight, or less than about 1.5% by weight, such as less than about 1% by weight of the ingredient to which it refers.

It is believed that one skilled in the art can, based upon the description herein, utilize the present invention to its fullest extent. The following specific embodiments are to be construed as merely illustrative, and not limitative of the remainder of the disclosure in any way whatsoever.

### Clay Portion

The clay portion of the composition comprises one or more clays. “Clay,” as used herein, refers to a class of hydrous silicate minerals that may finely divide in water so as to have an average (e.g., median) particle size of about 5 microns or less, such as about 2 microns or less. Clays are typically water insoluble, but are readily dispersible in water. Chemically, clays typically include silicon and oxygen as well as chemically bound water (hydrogen and oxygen), and may also include aluminum, magnesium, lithium, calcium, among other chemical moieties.

Clays suitable for use in compositions of the present invention may be single minerals or blends thereof, but the clay includes at least one bentonite.

“Bentonite” is an aluminum phyllosilicate that typically consists predominantly of montmorillonite and, as such, generally includes about 15% Al₂O₃ or more in its composition. In bentonite, the relative concentrations of sodium, potassium, calcium, as well as iron, magnesium and other elements may vary. The pH of bentonite in a 5% dispersion in deionized water may be from about 9 to about 10. One particularly suitable bentonite is VEEGUM HS, a high purity magnesium aluminum silicate commercially available from R.T. Vanderbilt of Norwalk, Conn.

In one embodiment, it is desirable that the clay portion comprise about 20 percent bentonite or more. According to another embodiment the clay portion consists essentially of bentonite. In another embodiment, the clay portion consists of bentonite.

For example, the amount of bentonite in the clay portion may be greater than about 95% percent.

According to certain embodiments of the invention, it may be desirable to include, in addition to bentonite, a second clay, in particular a synthetic hectorite clay. Synthetic hectorite clays may include lithium (e.g., about 0.5% or more of LiO₂) and may further include magnesium and sodium in their chemical structure. Synthetic hectorite clays are typically capable of achieving lower average particle sizes in solution than bentonite, (e.g., less than 1 micron) and are further capable of achieving disk-like shapes in water. One particularly suitable synthetic hecrorite is LAPONITE XLG, a lithium magnesium sodium silicate available from Southern Clay Products of Gonzales, Tex. In certain embodiments, the clay portion includes about 70 to about 80% by weight synthetic hectorite and about 20 to about 30% by weight bentonite.

The inventors have surprisingly found that by using a small amount of clay portion, the composition provides the critical balance of little to no flaking with a pleasant tightening sensation to the user.

In particular, the clay portion comprises about 0.75 to about 1.25% by weight of the composition, such as about 1% by weight of the composition.

### Polymer Particles

The composition includes polymer particles. Polymer particles suitable in the present invention are dispersible in water, with or without the assistance of a dispersing agent. According to certain embodiments, the polymer(s) that comprise the polymer portion have repeat units that include at least carbon and hydrogen, and optionally one or more of oxygen, silicon, nitrogen. According to certain embodiments, such polymer comprises a polyester or acrylic monomer. According to certain embodiments, the polymer comprising the polymer is crosslinked. According to certain other embodiments, the polymer particles are substantially spherical.

The inventors have found that polymer particles suitable in the present invention have a refractive index (such as may be measured at a wavelength of light from about 450 nm to about 500 nm) of about 1.3 to about 1.4, such as 1.30 to about 1.38.

Furthermore, the polymer particles have an average particle size (e.g., median particle size or median particle diameter) that is less than about 20 microns, say about 1 to about 20 microns. The average particle size of the polymer particles may be less than about 10 microns or less than about 8 microns. According to certain other embodiments, at least 90% of the polymer particles have a particle size in a range of about 0.5 to about 10 microns. According to certain embodiments, less than about 5% of the polymer particles have a size greater than 26 microns.

In one embodiment the polymer particles include a crosslinked polyester. One particularly suitable polyester is a polyester crosslinked with siloxane. The polyester may be formed by reacting a C4-C8 diacid with a branched C4-C6 diol, such as a polyester of adipic acid and neopentyl glycol crosslinked with isopropyltriethylsilane. A particular example of a suitable crosslinked polyester is a mixture of a polyester of adipic acid and neopentyl glycol crosslinked with isopropyltriethylsilane blended with a copolymer of vinyl pyrrolidone and vinyl acetate and is commercially available as AURASPHERE N (containing about 50% total polymer) from Centerchem, Inc. of Norwalk, Conn. The polymer particles of AURASPHERE N have a refractive index of 1.35 and a median particle size from about 0.5 to about 6 microns.

According to another embodiment, the polymer portion includes a crosslinked acrylic. One particularly suitable crosslinked acrylic is a crosslinked polymethyl methacrylate, e.g., a crosslinked polymethyl methacrylate powder. A particular example of a suitable crosslinked acrylic polymer is methmethacrylate crosspolymer available as SEPIMAT CPS (containing about 100% total polymer) from Seppic of Paris, France. SEPIMAT CPS has a median particle size of 4.5 to 8 microns and a refractive index of 1.33.

The amount of polymer particles in the composition is desirably about 1% to about 20%, such as about 2% to about 12%, such as about 2% to about 10%, such as about 2% to about 8%, for example about 2% to about 5%, by weight of the composition.

Furthermore, according to one embodiment, the weight ratio of polymer particles to clay portion (total amount of clays) is about 1:1 to about 10:1, such as about 2:1 to about 8:1, or about 2.5:1 to about 5:1.

The compositions of the present invention provide a balanced combination of pleasing sensations, both tactile and visual.

### Additional Polymers

The composition may further include additional polymers such those suitable for one or more of: enhancing the dispersion of the polymer particles in the composition, providing a gelling or thickening effect, or providing/enhancing film-formation. Examples of suitable additional polymers include synthetic polymers, in particular nonionic and/or cationic synthetic polymers. According to certain embodiments, the additional polymer has a weight average molecular weight of about 1000 to about 750,000.

Suitable synthetic polymers include homopolymers or copolymers such as those comprised of vinyl or acrylic monomers. Suitable vinyl polymers include copolymers of vinyl pyrrolidone, such as copolymers of vinyl pyrrolidone and vinyl acetate. Suitable acrylic polymers include polyacrylamides. An example of a suitable polyacrylamide is SEPIGEL 305, which includes about 45% polyacrylamide (and further includes laureth-7 and C13-C14 isoparaffin and water) and is commercially available from Seppic of Paris, France. Other suitable synthetic polymers are derivatized cellulose polymers. One suitable cellulose polymer is hydroxypropyl cellulose. Suitable cationic polymers include polymers derivatized with quaternium ammonium, e.g., polyquaternium-51.

While the composition may include natural polymers, in one embodiment, the composition is substantially free, or completely free, of polysaccharide polymers including: polysaccharide gums (e.g, natural polysaccharide gums such as xanthan gum, guar gum, carob gum and the like, such as those having a molecular weight of about 100,000 or greater, such as those having a molecular weight of about 200,000 or greater, such as those having a molecular weight 750,000 daltons or greater) and other natural or chemically modified polysaccharides or synthetic polysaccharides, such as chitosan, pectin, cationic and non-ionic cellulose polymers, and starches. In

In another embodiment, the composition is substantially free of natural polysaccharides gum polymers (such as those defined above), but may include other polysaccharides.

In another embodiment, the relative amount of polysaccharide polymers compared to the total amount of additional polymer in the composition is minimized.

For example, the total amount of polysaccharide polymers may be about 10% by weight or less, say less than about 5% by weight, of the total amount of additional polymers in the composition.

The total amount of additional polymers in the composition is desirably from about 0.5% to about 3%, such as from about 1% to about 2%, by weight of the composition.

### Humectant

According to one embodiment, the composition further includes one or more humectants. Humectants are hygroscopic and capable of hydrogen bonding with water and, according to certain embodiments the humectant includes at least two or more of the following functional groups: hydroxyl groups, amine/amino groups, and/or carboxylic acid groups. According to certain embodiments, the humectant has less than 3 carbon atoms per above functional group. Particularly suitable humectants include glycerin, butylene glycol, propylene glycol, urea, and trehalose. One notable ingredient that includes the humectants glycerin, sodium PCA (an amino acid) urea, and trehalose is ADVANCED MOISTURE COMPLEX available from BASF of Ludwigshafen, Germany.

The concentration of humectants may range from about 3% to about 20%, such as from about 4% to about 15%, such as from about 6 to about 15%, by weight of the composition.

Furthermore, according to one embodiment, the weight ratio of humectants to clay portion is about 5:1 to about 20:1, such as about 8:1 to about 15:1.

### Topical Composition

The composition of the present invention, aside from including clays, polymers and humectants, further includes water. The amount of water in the composition may be about 65% to about 97%, such as about 70% to about 90%, such as about 75% to about 85%, by weight of the composition.

The composition is substantially free of hydrophobic compounds. The composition may be completely free of hydrophobic compounds. As used herein, a “hydrophobic compound” means a compound that includes a hydrophobic moiety meeting one or more of the following three criteria: (a) has a carbon chain of at least ten carbons in which none of the ten carbons is a carbonyl carbon or has a hydrophilic moiety (defined below) bonded directly to it; (b) has two or more, preferably five or more alkyl siloxy groups and is free of hydrophilic moieties; or (c) has two or more oxypropylene groups in sequence. The hydrophobic moiety may include linear, cyclic, aromatic, saturated or unsaturated groups. The hydrophobic compound is not amphiphilic and, and such, does not include hydrophilic moieties. Hydrophilic moieties include anionic, cationic, zwitterionic, or nonionic group, that is polar, including sulfate, sulfonate, carboxylate, phosphate, phosphonates, ammonium, including mono-, di-, and trialkylammonium species, pyridinium, imidazolinium, amidinium, poly(ethyleneiminium), ammonioalkylsulfonate, ammonioalkylcarboxylate, amphoacetate, amine, amino, and poly(ethyleneoxy)sulfonyl moieties.

Examples of hydrophobic compounds that the composition may be substantially free of include C12 or greater oils such as vegetable oils (glyceryl esters of fatty acids, triglycerides), fatty esters, waxes, silicone oils, mineral oil and the like.

According to certain other embodiments, compositions of the present invention are substantially free of lower (e.g., C2-C3) monoalcohols such as ethanol and isopropanol.

Furthermore, compositions of the present invention may also be substantially free of color pigments, such as those typically used in color cosmetics (e.g., iron oxides, lake pigments, and interference pigments), to provide color to the skin.

Compositions of the present invention may otherwise include other functional ingredients such as anti-wrinkle agents, organic sunscreens, preservatives and fragrance. One example of a suitable preservative is EUXYL PE 9010, a mixture of phenoxyethanol and ethylhexylglycerin, available from Schulke & Mayr GmbH of Norderstedt, Germany.

According to certain embodiments, the pH of the composition is about 6.5 or greater, such as about 6.5 to about 8.5, such as about 7.5 to about 8.5, more particularly about 7.5 to about 8.5.

Compositions of the present invention are particularly suitable for topically applying to skin, such as for treating signs of aging, and, in particular, to under eye skin (e.g., generally between the eyeballs and the upper part of the cheekbone) in order to reduce the appearance of wrinkles while maintaining a pleasant sensory experience. The composition may be contained within or be in fluid communication with an applicator that is suitable for dispensing the product directly to the under eye skin.

### Example 1

Four compositions according to the invention, compositions E1, E2, E3 and E4, were prepared. The ingredients are shown below in Table 1.

TABLE 1

| | | Percent by Weight | | | |
|---|---|---|---|---|---|
| Ingredient | INCI | E1 | E2 | E3 | E4 |
| Purified | Water | 73.2 | 78.2 | 78.2 | 73.2 |
| Water | | | | | |
| Laponite | Lithium Magnesium Sodium | 0.75 | 0.75 | 0.75 | 0 |
| XLG | Silicate | | | | |
| Veegum HS | Magnesium Aluminum | 0.25 | 0.25 | 0.25 | 1.0 |
| Granules | Silicate | | | | |
| Glycerin | Glycerin | 2.0 | 2.0 | 2.0 | 2.0 |
| 99% USP | | | | | |
| Advanced | Glycerin, Sodium PCA, | 5.0 | 5.0 | 5.0 | 5.0 |
| Moisture | Urea, Polyquaternium 51; | | | | |
| Complex | Sodium hyaluronate; | | | | |
| | Thehalose; Chlorphensin; | | | | |
| | Phenoxyethanol; | | | | |
| | Methylparaben | | | | |
| Sepigel 305 | Polyacrylamide; Laureth-7; | 3.0 | 3.0 | 3.0 | 3.0 |
| | C13-C14 isoparaffin | | | | |
| 1,3 Butylene | Butylene glycol | 5.0 | 5.0 | 5.0 | 5.0 |
| glycol | | | | | |
| AuraSphere | Polyester of adipic acid and | 10.0 | 5.0 | | 10.0 |
| N | neopentyl glycol crosslinked | | | | |
| | with isopropyltriethylsilane | | | | |
| | blended with a copolymer of | | | | |
| | vinyl pyrrolidone and vinyl | | | | |
| | acetate | | | | |
| Sepimat CP5 | Methmethacrylate | | | 5.0 | |
| | crosspolymer | | | | |
| Euxyl PE | Phenoxyethanol; Ethylhexyl | 0.8 | 0.8 | 0.8 | 0.8 |
| 9010 | glycerin | | | | |

### Example 2

Compositions E1 and E4 were evaluated in a consumer test along with a comparative composition C1, YOUTHOLOGY NINETY SECOND WRINKLE REMOVING EYE SERUM, commercially available from YOUTHOLOGY Research Institute of Pacoima, California. The compositions were tested by 240 women of varying ethnicities, ages 30-60, and having concerns about bags, puffiness, dark circles, lines and wrinkles. The products were evaluated for overall likeability at 5 minutes, 30 minutes, 3 hours, and 1 week after applying them to under eye skin.

The percentage of respondents placing compositions E1 and E4 into the negative categories of “dislike extremely,” “dislike very much,” “dislike moderately,” or “dislike slightly” ranged from 3% to 12%. In contrast, the percentage of respondents placing composition C1 into the same negative categories was 20% to 24%. The difference in these percentage ranges was statistically significant.

According to its label, composition C1 did not contain polymer particles having an average particle size of less than about 20 microns and a refractive index from about 1.3 to about 1.4.

1. A composition, comprising:
water;
about 0.75% to about 1.25% by weight of a clay portion, wherein the clay portion comprises bentonite; and
polymer particles having an average particle size of less than about 20 microns and a refractive index of about 1.3 to about 1.4;
wherein the composition is completely free of hydrophobic compounds.

2. The composition of claim 1, wherein the clay portion comprises at least about 20% by weight bentonite.

3. The composition of claim 1, wherein the clay portion further comprises synthetic hectorite.

4. The composition of claim 1, wherein the polymer particles comprise a crosslinked polyester or a crosslinked acrylic.

5. The composition of claim 1, wherein the polymer particles comprise a polyester crosslinked with a siloxane.

6. The composition of claim 1 comprising about 2 to about 8% by weight of the polymer particles.

7. The composition of claim 1 further comprising an additional polymer having a weight average molecular weight of about 1000 to about 750,000.

8. The composition of claim 1, wherein the polymer particles have a refractive index of about 1.3 to about 1.38.

9. The composition of claim 1, comprising about 70 to about 90% by weight of water.

10. The composition of claim 1 substantially free of polysaccharide gums.

11. The composition of claim 1, wherein the weight ratio of the polymer particles to the clay portion is about 2:1 to about 8:1.

12. The composition of claim 1 having a pH of about 6.5 to about 8.5.

13. The composition of claim 1 having a pH of about 7 to about 8.

14. A composition, comprising:
about 70% to about 90% by weight of water;
about 0.75% to about 1.25% by weight of a clay portion, wherein the
clay portion comprises bentonite;
polymer particles having an average particle size of less than about 20 microns and a refractive index of about 1.3 to about 1.4; and
an additional polymer having a weight average molecular weight of about 1000 to about 750,000,
wherein said composition is completely free of hydrophobic compounds.

15. A method for treating under eye skin under the eyes comprising topically applying to said under eye skin a composition of claim 1.

16. (canceled)

17. The composition of claim 1 completely free of polysaccharide gums.

18. (canceled)

19. The composition of claim 14 completely free of polysaccharide gums.
